(19) United States
(12) Patent Application Publication
(10) Pub. No.: US 2013/0062429 A1
(43) Pub. Date: Mar. 14, 2013

(54) Decorative article

(71) Applicant: Hui Chin Vicki Chen, (US)

(72) Inventor: Hui Chin Vicki Chen, (US)

(21) Appl. No.: 13/199,825

(22) Filed: Sep. 9, 2011

(51) Int. Cl. A61L 9/04

(52) U.S. Cl. 239/34

(57) ABSTRACT

A decoration arrangement includes a supporting container, a predetermined amount of scent generating solution contained in the supporting container and a decorative article. The decorative article includes a supporting stem, a solution absorbing layer extended an outer surface of the supporting stem for absorbing the scent generating solution, a plurality of decorative elements spacedly extended from the supporting stem, and a plurality of connecting joints provided on the supporting stem to connect the decorative elements with the supporting stem, wherein the connecting joints is made of waterproof material so that the scent generating solution absorbed by the solution absorbing layer is prevented from being soaked by the decorative elements, so that a predetermined scent is generated by the decorative article without having the decorative elements to become wet.

### BACKGROUND OF THE PRESENT INVENTION

1. Field of Invention

The present invention relates to a decorative article, and more particularly to a decoration arrangement in which the decorative article is capable of generating a predetermined scene by using a scent generating solution without involving into excessive soaking thereof.

2. Description of Related Arts

A conventional decorative article such as a candle generating a predetermined kind of fragrance usually comprises a candle body soaked with a predetermined amount of perfume so as to allow the candle to generate a very pleasant scent when a user is using the candle. On the other hand, other kinds of decorative articles also exist. For example, an artificial plant may be immersed into a predetermined kind of perfume so that when it is placed in a desirable place, the plant will produce a specific scent for leisure.

There are several disadvantages in associated with these conventional decorative articles. First, the perfume as used in the decorative article may come into contact with users or other objects in a domestic environment and therefore may cause damage to those objects. For example, when perfume comes into contact with the hand of a kid who subsequent rub his eyes with the hands, the perfume may cause harm to the kid's eyes. Second, the decorative article may require a large amount of perfume which may cause fire when the decorative article is put near a stove or other fire generating devices. Third, conventional artificial plants usually have very poor liquid absorbing ability so that there is virtually no scent coming out from the top potion of the plants.

### SUMMARY OF THE PRESENT INVENTION

The invention is advantageous in that it provides a decoration arrangement in which the decorative article is capable of generating a predetermined scene by using a scent generating solution without involving into excessive soaking thereof.

Another advantage of the invention is to provide a decorative arrangement which is capable of uniformly generating a predetermined scent along the entire supporting stem of a decorative article so as to overcome the problem of inadequate or non-uniform scent generation along a vertical direction of the conventional decorative article.

Another advantage of the invention is to provide a decorative arrangement in which the decorative article can be assembled and disassembled easily and conveniently so as to allow widespread application of the present invention.

Another advantage of the invention is to provide a decorative arrangement in which the decorative article comprising a plurality of connecting joints which are capable of blocking the scent generating solution from reaching the decorative elements.

Additional advantages and features of the invention will become apparent from the description which follows, and may be realized by means of the instrumentalities and combinations particular point out in the appended claims.

According to the present invention, the foregoing and other objects and advantages are attained by providing a decoration arrangement, comprising:

a supporting container having a receiving cavity formed therein;

a predetermined amount of scent generating solution contained in the supporting container; and

a decorative article, which comprises:

a supporting stem having a lower end portion immersed in the scent generating solution in the supporting container, and a main portion extended upwardly from the lower end portion to expose out of the supporting container;

a solution absorbing layer extended an outer surface of the supporting stem for absorbing the scent generating solution;

a plurality of decorative elements spacedly extended from the main portion of the supporting stem; and

a plurality of connecting joints provided on the supporting stem to connect the decorative elements with the supporting stem, wherein the connecting joints is made of waterproof material so that the scent generating solution absorbed by the solution absorbing layer is prevented from being soaked by the decorative elements, so that a predetermined scent is generated by the decorative article without having the decorative elements to become wet.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a side view of a decoration arrangement according to a preferred embodiment of the present invention.

FIG. 2 is a sectional side view of the decoration arrangement according to the above preferred embodiment of the present invention.

FIG. 3 is a schematic diagram of the decoration arrangement according to a preferred embodiment of the present invention.

### DETAILED DESCRIPTION OF THE PREFERRED EMBODIMENT

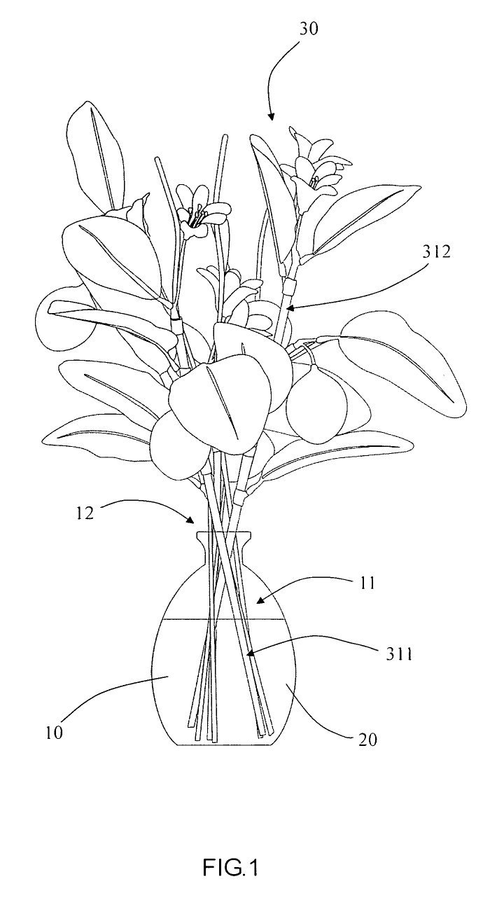

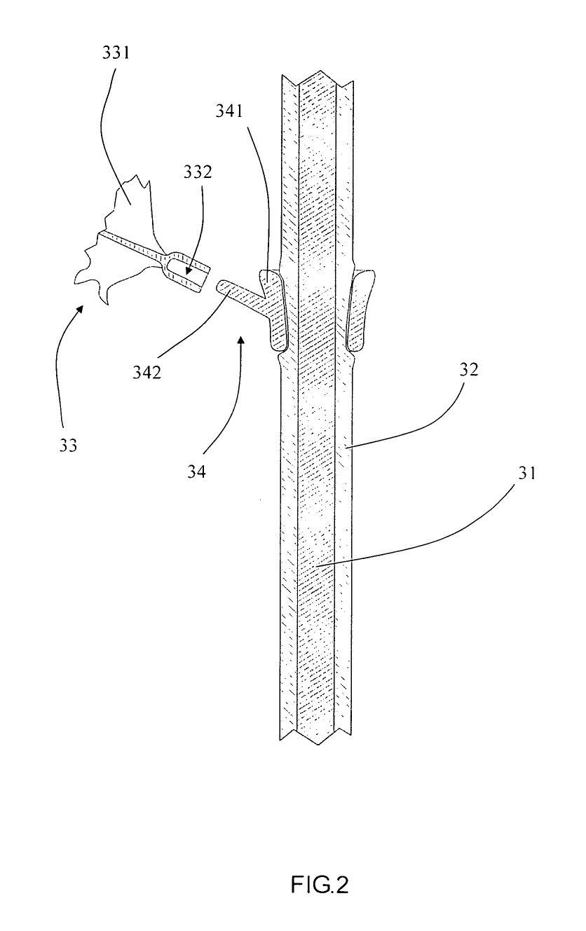

Referring to FIG. 1 to FIG. 3 of the drawings, a decoration arrangement according to a preferred embodiment of the present invention, in which the decoration arrangement comprises a supporting container 10 having a receiving cavity 11 and an opening 12 communicating the receiving cavity 11 with an exterior of the supporting container 10, a predetermined amount of scent generating solution 20 contained in the supporting container, and a decorative article 30.

The decorative article 30 comprises a supporting stem 31, a solution absorbing layer 32, a plurality of decorative elements 33, and a plurality of connecting joints 34. The supporting stem 31 has a lower end portion 311 immersed in the scent generating solution 20 in the supporting container 10, and a main portion 312 extended upwardly from the lower end portion 311 to expose out of the supporting container 10.

The solution absorbing layer 32 is extended an outer surface of the supporting stem 31 for absorbing the scent generating solution 20. The plurality of decorative elements 33 is spacedly extended from the main portion 312 of the supporting stem 31.

The plurality of connecting joints 34 is provided on the supporting stem 31 to connect the decorative elements 33 with the supporting stem 31, wherein the connecting joints 34 are made of waterproof material so that the scent generating solution 20 absorbed by the solution absorbing layer 32 is prevented from being soaked by the decorative elements 33, so that a predetermined scent is generated by the decorative article without having the decorative elements to become wet.

According to the preferred embodiment of the present invention, the supporting container 10 has a predetermined aesthetic appearance so as to fit its aesthetic style with that of the decorative article 30. For example, the supporting container 10 may be embodied as a vast wherein the decorative article 30 is shaped and crafted to resemble a bundle of flowers or other plants. In this case, the supporting container 10 is preferably made of transparent material such as glass so that the scent generating solution 20 can be seen from an exterior of the supporting container 10.

The scent generating solution 20 is stored in the receiving cavity 11 of the supporting container 10 and is arranged to generate a predetermined scent or smell so that when it is absorbed by the decorative article 30, the decorative article is arranged to generate that scent to the ambient environment. The scent generation solution 20 may have a predetermined color for additional aesthetic effect.

The supporting stem 31 of the decorative article 30 is impermeable to liquid so that the scent generating solution 20 is not capable of being absorbed by the supporting stem 31. Rather, the scent generating solution 20 is arranged to be absorbed by the solution absorbing layer 32 which embed the supporting stem 31 along its entire length. As such, the scent generating solution 20 is capable of reaching the entire length of the supporting stem 31 for uniform generation of the predetermined scent.

The connecting joints 34 are spacedly provided along the solution absorbing layer 31 and are extended therefrom, wherein each of the connecting joints 34 is liquid impermeable so that the scent generating solution 20 is incapable of reaching the decorative elements 33 from the solution absorbing layer 32 because the scent generating solution 20 is blocked by the connecting joints 34.

Each of the decorative elements 33 comprises an aesthetic structure 331 having a connecting slot 332 formed thereon, whereas each of the connecting joints 34 has a main portion 341 attached on the solution absorbing layer 32 of the corresponding decorative element 33 and a connector shaft 342 extended form the main portion 341 and is arranged to detachably insert into the connecting slot 332 of the corresponding decorative element 33 so as to connect that corresponding decorative element 33 with the supporting stem 31 of the decorative article 30.

The aesthetic structure 331 of the decorative element 33 may comprise an artificial leaf and/or an artificial flower which are shaped and crafted to resemble a genuine leaf or flower. Since an artificial flower or leaf does not have scent which usually comes out from a genuine flower, the scent generating solution 20 as absorbed by the solution absorbing layer 32 fulfills that feature. Moreover, since the scent generating solution 20 does not reach the decorative elements 33, when a user comes to observe the decorative article 30 and touches the decorative elements 33, his or her hands will not touch the scent generating solution 20 which may be harmful to the user on physical contact, especially for children.

According to the preferred embodiment of the present invention, the main portion 341 of each of the connecting joints 34 is tubular in shape in which a diameter of an upper part of the main portion 341 is slightly larger than that of a lower part of the main portion 341, wherein the supporting stem 31 and the solution absorbing layer 32 are inserted into the main portion 341 of the connecting joints 34 so as to attach the connecting joints 34 onto the supporting stem 31. Since the diameter of the upper part of the connecting joint 34 is slightly larger than its lower part, this structure helps in securely attaching the connecting joints 34 onto the supporting stem 11.

The solution absorbing layer 32 of the decorative article 30 is preferably made of liquid absorbing material, such as paper material, fabric material, cotton material, or the likes. These materials are very good liquid absorbents but are easily attachable on the supporting stem 31. Moreover, these materials are easily and conveniently cut so as to allow manufacturer of the present invention to cut the solution absorbing layer 32 to be tailored-made for specific decorative elements 33.

It is also worth mentioning that each of the supporting stems 31 of the decorative article 30 is freely bendable in the sense that a user is able to adjust the shape of the supporting stem 31 by manually bending the corresponding supporting stem 31 into a desirable shape.

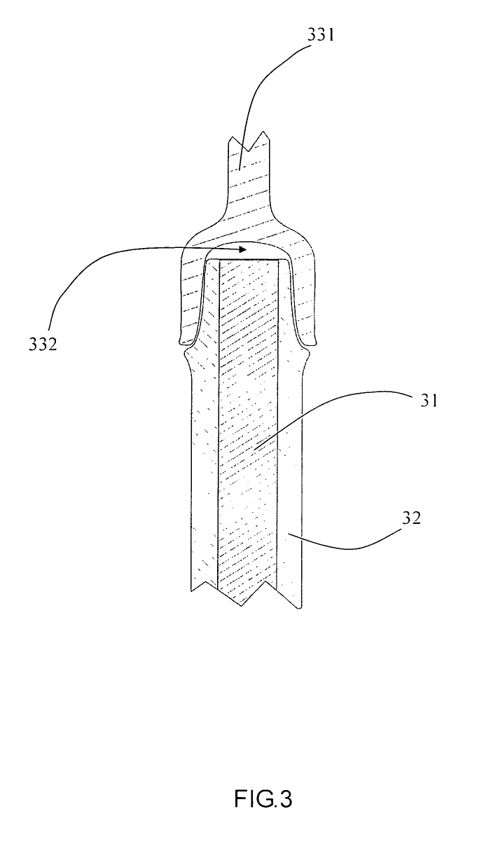

As shown in FIG. 3 of the drawings, each of the decorative elements may also be directly coupled to the supporting stem 31 and the liquid absorbing layer 32 without using the connecting joint 34. In this scenario, the supporting stem 31 and the liquid absorbing layer 32 are detachably inserted into the connecting slot 332 of the aesthetic structure 331. This structure enables the aesthetic structure 331 which is positioned on top of the supporting stem 31 to be conveniently and detachably attached thereonto without the hassle of using the connecting joint 34.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A decoration arrangement, comprising:
a supporting container having a receiving cavity formed therein;
a predetermined amount of scent generating solution contained in said supporting container; and
a decorative article, which comprises:
a supporting stem having a lower end portion immersed in said scent generating solution in said supporting container, and a main portion extended upwardly from said lower end portion to expose out of said supporting container;
a solution absorbing layer extended an outer surface of said supporting stem for absorbing said scent generating solution;
a plurality of decorative elements spacedly extended from said main portion of said supporting stem; and
a plurality of connecting joints provided on said supporting stem to connect said decorative elements with said supporting stem, wherein said connecting joints is made of waterproof material so that said scent generating solution absorbed by said solution absorbing layer is prevented from being soaked by said decorative elements, so that a predetermined scent is generated by said decorative article without having said decorative elements to become wet.

2. The decorative article, as recited in claim 1, wherein said scent generating solution is replaceably stored in said receiving cavity of said supporting container and is arranged to generate a predetermined scent so that when said scent generating solution is absorbed by said decorative article, said decorative article is arranged to generate that scent to said ambient environment.

3. The decorative article, as recited in claim 2, wherein said scent generating solution is arranged to be absorbed by said solution absorbing layer which embeds said supporting stem along an entire length thereof, such that said scent generating solution is capable of reaching said entire length of said supporting stem for uniform generation of said predetermined scent.

4. The decorative article, as recited in claim 3, wherein said connecting joints are spacedly provided along said solution absorbing layer and are extended therefrom, wherein each of said connecting joints is liquid impermeable so that said scent generating solution is incapable of reaching said decorative elements from said solution absorbing layer as said scent generating solution is blocked by said connecting joints.

5. The decorative article, as recited in claim 4, wherein each of said decorative elements comprises an aesthetic structure having a connecting slot formed thereon, whereas each of said connecting joints has a main portion attached on said solution absorbing layer of said corresponding decorative element and a connector shaft extended from said main portion and is arranged to detachably insert into said connecting slot of said corresponding decorative element so as to connect that corresponding decorative element with said supporting stem of said decorative article.

6. The decorative article, as recited in claim 2, wherein said main portion of each of said connecting joints is tubular in shape in which a diameter of an upper part of said main portion is slightly larger than that of a lower part of said main portion, wherein said supporting stem and said solution absorbing layer are inserted into said main portion of said connecting joints so as to attach said connecting joints onto said supporting stem.

7. The decorative article, as recited in claim 3, wherein said main portion of each of said connecting joints is tubular in shape in which a diameter of an upper part of said main portion is slightly larger than that of a lower part of said main portion, wherein said supporting stem and said solution absorbing layer are inserted into said main portion of said connecting joints so as to attach said connecting joints onto said supporting stem.

8. The decorative article, as recited in claim 4, wherein said main portion of each of said connecting joints is tubular in shape in which a diameter of an upper part of said main portion is slightly larger than that of a lower part of said main portion, wherein said supporting stem and said solution absorbing layer are inserted into said main portion of said connecting joints so as to attach said connecting joints onto said supporting stem.

9. The decorative article, as recited in claim 6, wherein each of said supporting stems of said decorative article is freely bendable so that a user is able to adjust a shape of said supporting stem by manually bending said corresponding supporting stem into a desirable shape.

10. The decorative article, as recited in claim 7, wherein each of said supporting stems of said decorative article is freely bendable so that a user is able to adjust a shape of said supporting stem by manually bending said corresponding supporting stem into a desirable shape.

11. The decorative article, as recited in claim 8, wherein each of said supporting stems of said decorative article is freely bendable so that a user is able to adjust a shape of said supporting stem by manually bending said corresponding supporting stem into a desirable shape.

12. The decorative article, as recited in claim 5, wherein said supporting stem of said decorative article is impermeable to liquid so that said scent generating solution is not capable of being absorbed by said supporting stem.

13. The decorative article, as recited in claim 8, wherein said supporting stem of said decorative article is impermeable to liquid so that said scent generating solution is not capable of being absorbed by said supporting stem.

14. The decorative article, as recited in claim 11, wherein said supporting stem of said decorative article is impermeable to liquid so that said scent generating solution is not capable of being absorbed by said supporting stem.

15. The decorative article, as recited in claim 8, wherein said solution absorbing layer of said decorative article is made of liquid absorbing material.

16. The decorative article, as recited in claim 11, wherein said solution absorbing layer of said decorative article is made of liquid absorbing material.

17. The decorative article, as recited in claim 14, wherein said solution absorbing layer of said decorative article is made of liquid absorbing material.

18. The decorative article, as recited in claim 11, wherein said supporting container is transparent in color and is embodied as a vast having a predetermined aesthetic appearance so as to fit an aesthetic style of said decorative article, wherein said supporting container further has a top opening communicating said receiving cavity with an exterior of said supporting container.

19. The decorative article, as recited in claim 14, wherein said supporting container is transparent in color and is embodied as a vast having a predetermined aesthetic appearance so as to fit an aesthetic style of said decorative article, wherein said supporting container further has a top opening communicating said receiving cavity with an exterior of said supporting container.

20. The decorative article, as recited in claim 17, wherein said supporting container is transparent in color and is embodied as a vast having a predetermined aesthetic appearance so as to fit an aesthetic style of said decorative article, wherein said supporting container further has a top opening communicating said receiving cavity with an exterior of said supporting container.
